(19) United States
(12) Patent Application Publication
(10) Pub. No.: US 2015/0259311 A1
(43) Pub. Date: Sep. 17, 2015

(54) METHOD OF PRODUCING LACTONE FROM HYDROXYCARBOXYLIC ACID OR DICARBOXYLIC ACID IN AQUEOUS SOLUTION

(71) Applicant: Samsung Electronics Co., Ltd., Suwon-si (KR)

(72) Inventors: Kyunghae Lee, Incheon (KR); Mooho Lee, Suwon-si (KR); Jun Chwae, Seoul (KR); Jinhwan Park, Suwon-si (KR); Jongmin Lee, Hwaseong-si (KR); Kwangmyung Cho, Seongnam-si (KR); Jaechan Park, Yongin-si (KR)

(21) Appl. No.: 14/656,427

(22) Filed: Mar. 12, 2015

(30) Foreign Application Priority Data
Mar. 12, 2014 (KR) 10-2014-0029272
Mar. 6, 2015 (KR) 10-2015-0031799

(51) Int. Cl. C07D 307/33

(52) U.S. Cl. CPC C07D 307/33

(57) ABSTRACT

A method of producing lactone by converting an aqueous solution including hydroxycarboxylic acid or dicarboxylic acid to lactone under an acid condition and in the presence of a water immiscible solvent.

### RELATED APPLICATION

This application claims the benefits of Korean Patent Application No. 10-2014-0029272, filed on Mar. 12, 2014, and Korean Patent Application No. 10-2015-0031799, filed on Mar. 6, 2015, in the Korean Intellectual Property Office, the entire disclosure of which is hereby incorporated by reference.

### BACKGROUND

1. Field

The present disclosure relates to a method of producing lactone from hydroxycarboxylic acid or dicarboxylic acid in an aqueous solution.

2. Description of the Related Art

Lactone is a compound used in manufacturing industries, for instance, as an intermediate in pharmaceutical, fine chemical, or agricultural chemical production. Also, lactone may be used as a solvent or a monomer in polymer technologies.

Lactone has been produced through a chemical synthesis process using hydroxycarboxylic acid or dicarboxylic acid, but an increase in oil prices raises the cost of producing lactone by such methods. Thus, a process of producing lactone by an alternate chemical synthesis process is needed.

### SUMMARY

Provided is a method of producing lactone comprising adding an acid to an aqueous solution comprising a hydroxycarboxylic acid or dicarboxylic acid to adjust a pH of the aqueous solution to 4 or lower, whereby the so that hydroxycarboxylic acid or dicarboxylic acid is converted to a lactone; adding a water immiscible solvent to the aqueous solution to distribute the lactone to a solvent layer; and collecting the lactone from the solvent layer.

Additional aspects will be set forth in part in the description which follows and, in part, will be apparent from the description, or may be learned by practice of the presented exemplary embodiments.

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms, and the invention should not be construed as being limited to the descriptions set forth herein. Accordingly, the exemplary embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

According to an aspect of an exemplary embodiment, a method of producing lactone includes adding an acid to an aqueous solution including hydroxycarboxylic acid or dicarboxylic acid to adjust the pH of the aqueous solution to 4 or lower so that hydroxycarboxylic acid or dicarboxylic acid is converted to lactone; adding a water immiscible solvent to the aqueous solution to distribute the lactone to a solvent layer; and collecting the lactone from the solvent layer.

In the method, the aqueous solution may be a culture solution of one or more microorganisms including hydroxycarboxylic acid or dicarboxylic acid. Thus, the method may further include culturing microorganisms that produce hydroxycarboxylic acid or dicarboxylic acid to produce a culture solution including hydroxycarboxylic acid or dicarboxylic acid. The microorganisms may include bacteria such as E. Coli which belongs to genus Escherichia or genus Corynebacterium. The culturing of the microorganisms may be performed under neutral or weak acidic condition. The culturing of the microorganisms may be performed at a pH in a range of about 6 to about 7.

The method may include removing a solid from the culture. The removing of the solid may be performed by centrifuge, filtration, precipitation, or a combination thereof. The method may further include, for example, filtering the culture solution to remove retentate. The filtering of the culture solution may remove microorganism cells, proteins or some mineral salts from the culture solution.

Also, the method may further include heating of the aqueous solution including the solvent after the adding of the solvent. The heating of the aqueous solution may be performed at a temperature lower than a boiling point of the solvent, for example, about 10° C. lower than the boiling point of the solvent, or about 15° C. lower than the boiling point of the solvent. The heating of the aqueous solution may be performed at a temperature in a range of about 50° C. to about 90° C., about 50° C. to about 80° C., about 50° C. to about 70° C., or about 50° C. to about 60° C. A period of time for the heating of the aqueous solution may be appropriately determined in consideration of a degree of converting the hydroxycarboxylic acid or dicarboxylic acid to lactone.

As used herein, the term “hydroxycarboxylic acid” or “dicarboxylic acid” may refer to a free acid form thereof, an anion form thereof, or a salt thereof that may be used alternatively.

The hydroxycarboxylic acid may have a structure of Formula 1:

HO—R1-COOH (Formula 1)

In Formula 1, R1 is a linear or branched substituted or unsubstituted C1-C20 alkyl group. R1 may be a linear C1-C15, C1-C10, C1-C8, C2-C10, or C2-C8 alkyl group. The substituted alkyl group may be substituted with at least one halogen. The hydroxycarboxylic acid may include —OH group at a carbon at a location of 2, 3, 4, 5, or 6 in Formula 1, wherein locations 2, 3, 4, 5 or 6 correspond to a carbon position (numbering of carbons) counted from a carbon of the carbonyl group in the carboxyl group, e.g., the carbonyl group carbon of the carboxy group is position 1. The shortest carbon chain length is followed if R1 is a branched alkyl group. R1 may be a linear C1-C10 alkyl group. The hydroxycarboxylic acid may be 3-hydroxypropionic acid, 4-hydroxybutyric acid, 5-hydroxypentanic acid, or 6-hydroxyhexanoic acid.

The dicarboxylic acid may have a structure of Formula 2:

HOOC—R2-COOH (Formula 2)

In Formula 2, R2 is absent (e.g., R2 is a bond) or is a linear or branched substituted or unsubstituted C1-C20 alkyl group. R2 may be a linear C1-C15, C1-C10, C1-C8, C2-C10, or C2-C8 alkyl group. The substituted alkyl group may be substituted with at least one halogen. The dicarboxylic acid may have 0, 1, 2, 3, or 4 carbons in a shortest carbon chain that links —COOH group and —COOH group in Formula 2, and R2 is either absent (e.g., R2 is a bond) or a linear C1-C8 alkyl group. The dicarboxylic acid may be succinic acid, 2-ethylsuccinic acid, glutaric acid, 2-methylglutaric acid, 2-ethylglutaric acid, adipic acid, 2-methyladipic acid, 3-methyladipic acid, 4-methyladipic acid, 5-methyladipic acid, 2,2-dimethyladipic acid, 3,3-dimethyladipic acid, 2,2,5-trimethyladipic acid, 2,5-dimethyladipic acid, pimelic acid, 2-methylpimelic acid, 2,2-dimethylpimelic acid, 3,3-dimethylpimelic acid, 2,2,5-trimethylpimelic acid, azelic acid, or sebacic acid.

The method further includes adding an acid to an aqueous solution including hydroxycarboxylic acid or dicarboxylic acid to adjust a pH of the aqueous solution to 4 or lower so that hydroxycarboxylic acid or dicarboxylic acid is converted to lactone. The acid added to the aqueous solution to adjust the pH may be an organic acid or an inorganic acid. The acid may be sulfuric acid, phosphoric acid, or hydrochloric acid. Acid having pKa that is lower than pKa of the hydroxycarboxylic acid or dicarboxylic acid may be added to the aqueous solution. Since the hydroxycarboxylic acid or dicarboxylic acid may be present in the aqueous solution (e.g., culture solution) as a salt as well as free acid, an acid that corresponds to a salt present in the aqueous solution may be formed by addition of the lower pKa acid. The pKa of the acid that is added to the aqueous solution to lower the pH may be, for example, 7 or lower, 4 or lower, 3 or lower, 2 or lower, 1 or lower, or 0.5 or lower. For example, when 4-hydroxybutyric acid (4-HB) is present in the aqueous solution as a 4-hydroxybutyrate salt, 4-hydroxybutyric acid may be formed by adding an acid having pKa lower than pKa of 4-hydroxybutanic acid, such as, sulfuric acid or hydrochloric acid. After adding the acid, the pH of the aqueous solution may be about 4 or lower (e.g., about 0.5 to about 4, about 2 to about 3.5, or about 2 to about 4); about 3 or lower (e.g., about 2 to about 3, or about 2 to about 2.5), about 2 or lower (e.g., about 1 to about 0.5), or about 1 or lower.

The hydroxycarboxylic acid or dicarboxylic acid may form a lactone by internal cyclization. The internal cyclization may form lactone from carboxylic acid through dehydration. The internal cyclization may be reversible reaction. The internal cyclization may be, for example, a conversion reaction from 4-HB to γ-butyrolactone.

In the method, the term “lactone” denotes a compound including an ester group in a ring. The lactone may be gamma-butyrolactone (γ-butyrolactone: GBL), β-propiolactone, δ-valerolactone, ε-caprolactone, α-angelica lactone, β-angelica lactone, or γ-valerolactone (GVL). The lactone may be converted to at least one of 1,4-butandiol (BDO), tetrahydrofuran (THF), N-methylpyrrolidone (NMP), N-ethylpyrrolidone (NEP), 2-pyrrolidone, N-vinylpyrrolidone (NVP), and polyvinylpyrrolidone (PVP).

The method may include distributing the lactone to a solvent layer by adding a water immiscible solvent to the aqueous solution.

The water solubility of the water immiscible solvent may be about 2.00 g/dL or lower, for example, about 0.05 g/dL to about 2.00 g/dL, about 0.05 g/dL to about 1.50 g/dL, or about 0.70 g/dL to about 1.50 g/dL, as measured at 20° C. to 25° C. and 1 atm. The water immiscible solvent may be a polar solvent or a polar aprotic solvent. The water immiscible solvent may be chlorobenzene, dichloromethane, or chloroform. The water immiscible solvent may be added to the aqueous solution and form two phases including an aqueous phase and a solvent layer, which is an organic phase. When lactone is produced from hydroxycarboxylic acid or dicarboxylic acid in the aqueous phase, the produced lactone has a higher solubility with respect to the water immiscible solvent than to the aqueous solution. Thus, the produced lactone may dissolve in the water immiscible solvent. Production of lactone from hydroxycarboxylic acid or dicarboxylic acid is reversible, and the forward reaction producing lactone is inhibited by increasing concentration of lactone in the aqueous phase. Thus, as the lactone is removed from the aqueous phase, conversion of lactone from hydroxycarboxylic acid or dicarboxylic acid may be accelerated.

Any suitable amount of the water immiscible solvent may be used, for example, about 0.5 to about 3.0 fold, about 0.5 to about 2.0 fold, about 1.0 to about 2.0 fold, or about 1.5 to about 2.0 fold larger than the volume of the aqueous solution. As the volume of the water immiscible solvent increases, lactone may better dissolve in the water immiscible liquid, and a greater amount of lactone may be collected.

The method includes the collecting (e.g., separating or isolating) of the lactone from the solvent layer. The collecting may be performed by collecting the solvent layer and evaporating the solvent from the solvent layer to separate lactone. The lactone may be collected based on a difference between the boiling points of the water immiscible solvent and lactone. The water immiscible solvent may be added back to the aqueous solution for re-use after the lactone is removed from the solvent.

In the method, “adding an acid to an aqueous solution comprising hydroxycarboxylic acid or dicarboxylic acid to adjust a pH of the aqueous solution to 4 or lower so that the hydroxycarboxylic acid or the dicarboxylic acid is converted to lactone” may be “adding an acid to a culture solution comprising 4-hydrobutyric acid to adjust a pH of the aqueous solution to 4 or lower so that the 4-hydrobutyric acid is converted to GBL”; and “adding a water immiscible solvent to the aqueous solution to distribute the lactone to a solvent layer” may be “adding dichloromethane or chloroform to the culture solution to distribute the GBL to a chloromethane or chloroform layer. In one embodiment, the method may further include heating of the aqueous solution to a temperature in a range of about 50° C. to about 70° C. In one embodiment, pH of the aqueous solution may be in a range of about 0.50 to about 2.00.

The present invention will be described in further detail with reference to the following examples. These examples are for illustrative purposes only and are not intended to limit the scope of the present invention

### Example 1

### Conversion of 4-HB to GBL Under Acid Condition in the Presence of Solvent

In the present embodiment, an effect of an acid condition and the presence of a solvent on conversion or extraction of GBL from 4-HB was confirmed.

1. Conversion of 4-HB to GBL Under Acid Condition

First, the effect of acid concentration (pH) on the conversion of 4-HB to GBL was analyzed.

E. Coli producing 4-HB was cultured in a medium (13.5 g KH₂PO₄, 4 g (NH₄)₂HPO₄, 1.7 g citric acid, 1.4 g MgSO₄.7H₂O, Yeast extract 2 g, MOPS 21 g, 10 mL trace metal solution (10 g FeSO₄.7H₂O, 1.35 g CaCl₂, 2.25 g ZnSO₄.7H₂O, 0.5 g MnSO₄.4H₂O, 1 g CuSO₄.5H₂O, 0.106 g (NH₄)₆Mo₇O₂₄.4H₂O, 0.23 g Na₂B₄O₇.10H₂O, 35% HCl 10 mL per 1 L distilled water) per 1 L distilled water) for 200 hours to obtain 9˜12 (w/v) % 4-HB containing culture solution (pH about 6˜7.0). 95 (w/v) % sulfuric acid was added to 100 ml of the culture solution so that pH of the culture solution was 0.5, 1.0, 2.0, or 4.0 in each of the 100 ml of the culture solution as a sample. The culture solution was maintained at room temperature for 1 hour to allow 4-HB to convert to GBL. After 1 hour, each of the samples was placed in HPLC (Waters, Column: C18, Room Temperature (25° C.), UV: 195 nm, Flow: 1.0 ml/min, Injection volume: 2 μL, Buffer: 25 mM KH₂PO₄) and GC (Younglin G C, Detector: FID) to measure 4-HB and GBL. Table 1 shows the effect of pH on conversion of 4-HB to GBL.

TABLE 1

| | pH | | | |
|---|---|---|---|---|
| | 4 | 2 | 1 | 0.5 |
| 4-HB concentration | 10.15 | 9.73 | 10.63 | 9.94 |
| (w/v %) | | | | |
| GBL yield (%) | 2 | 16 | 54 | 68 |

In Table 1, GBL yield (%)={[1−(a 4-HB concentration after the reaction was completed (g/L)/a 4-HB concentration in a solution before the reaction (g/L)]×(a molecular weight of GBL/a molecular weight of 4-HB)}×100.

As shown in Table 1, when pH is lowered, a GBL yield increased. This is because when pH is lowered as acid is added, the conversion of GBL from 4-HB is accelerated in the solution.

2. Extraction of GBL According to Solvent

Next, the effect of a water immiscible solvent, such as a non-polar solvent or a polar aprotic solvent, on the extraction of GBL was analyzed.

In particular, 100 ml aliquots of the 9˜12 (w/v) % 4-HB containing culture solution (pH about 6˜7.0) from step 1 were mixed with 100 ml of a water immiscible solvent, each aliquot being combined a different solvent. The mixture was maintained for 1 hour so that GBL was distributed into a solvent layer. Then, the solvent layer was separated from the mixture, and a sample from the solvent layer was placed in GC (Younglin G C, Detector: FID) to measure an amount of GBL in the sample. Table 2 shows the extraction ratios of GBL according to different water immiscible solvent.

TABLE 2

| Solvent | Water solubility(g/dL)* | Extraction ratio (%) |
|---|---|---|
| Heptane | 0.01 | 0 |
| Hexane | 0.014 | 0.9 |
| Diethylether | 6.9 | 20.96 |
| Para-xylene | Insoluble | 23.01 |
| Toluene | 0.05 | 33.2 |
| Cholorobenzene | 0.05 | 40.6 |
| Dichloromethane | 1.32 | 71.74 |
| Chloroform | 0.795 | 72.5 |

*water solubility represents water weight (g) solubilized in 100 mL solvent measured at 20° C. to 25° C. and 1 atm.

In Table 2, extraction ratio (%)={(a GBL concentration in a solvent layer (g/L)×a volume of the solvent layer (L))/(a GBL concentration in a solution before the reaction (g/L)×a volume of the solution before the reaction (L)}×100.

As shown in Table 2, solvents having a water solubility in a water immiscible solvent in a range of about 0.05 g/dL to about 1.5 g/dL, for example, about 0.7 g/dL to about 1.5 g/dL, which were toluene, chlorobenzene, dichloromethane, and chloroform, provided good extraction. Dichloromethane and chloroform, having water solubility of about 0.8 g/dL or greater, provided particularly high extraction ratios of GBL compared to other solvents.

Also, effect of the amount of a solvent on extraction of GBL was confirmed. One of the solvents with high extraction ratios of GBL, chloroform, was used by varying a volume of the solvent in a range of about 0.5 to about 2 folds of a volume of the aqueous culture solution. In particular, chloroform was added to 100 ml of the 9˜12 (w/v) % 4-HB containing culture solution (pH about 6˜7.0) of step 1 at a volume of about 0.5 to about 2 fold, that is, about 0.50, 0.97, 1.51, or 1.84 fold volume of the culture solution to extract GBL. Table 3 shows the results of GBL extraction ratios (%) thus measured.

TABLE 3

| | Fold-volume of added chloroform | | | |
|---|---|---|---|---|
| | 0.50 | 0.97 | 1.51 | 1.84 |
| Extraction ratio (%) | 57.8 | 72.5 | 74.95 | 83.50 |

As shown in Table 3, when an amount of chloroform increased, an extraction ratio of GBL increased.

3. Conversion and Extraction of 4-HB to GBL Under Acid Condition in the Presence of Water Immiscible Solvent

95% sulfuric acid was added to 100 ml of the 9˜12 (w/v) % 4-HB containing culture solution (pH about 6˜7.0) of step 1 until pH of the solution was about 1 or about 0.5 in each sample. 100 ml of chloroform, as an extraction solvent, was added thereto. The mixture was stirred at room temperature for 1 hour to allow conversion of 4-HB to GBL. As a control group, 100 ml samples were prepared in the same manner described above, except that chloroform was not used in the control group.

When the reaction was completed, a sample was obtained from each solvent layer and aqueous layer, and the samples were used to measure amounts of 4-HB and GBL in the same manner described in Example 1. From the results, extraction ratios (%), and solvent conversion yields (%) were calculated.

Solvent conversion yield=a GBL yield (%)×an extraction ratio (%)/100.

Table 4 shows the calculated extraction ratios (%), and solvent conversion yields (%).

TABLE 4

| | | Amount of | Initial | | Extrac- | Solvent |
|---|---|---|---|---|---|---|
| | | solvent | 4-HB | GBL | tion | con- |
| | | added | concen- | yield | ratio | version |
| | pH | (fold) | tration | (%) | (%) | yield (%) |
| Experimental | 1 | 1.00 | 11.23 | 58 | 68 | 40 |
| group 1 | | | | | | |
| Control group 1 | 1 | 0 | 10.63 | 54 | — | — |
| Experimental | 0.5 | 1.00 | 8.27 | 82 | 73 | 60 |
| group 2 | | | | | | |
| Control group 2 | 0.5 | 0 | 9.94 | 68 | — | — |

As shown in Table 4, when dehydration was performed by adding a solvent, conversion of 4-HB to GBL increased. This is because GBL formed by adding a water immiscible solvent such as chlorobenzene, dichloromethane, or chloroform that has a high solubility in the water immiscible solvent compared to that of an aqueous solution. Thus GBL is included in the water immiscible solvent. Also, as a concentration of GBL decreases in the aqueous solution, the conversion of 4-HB to GBL is accelerated.

### Example 2

### Conversion and Extraction of GBL from 4-HB by Heating Under Acid Condition in the Presence of Solvent

A reaction was performed in the same manner as used in step 3 of Example 1, except that chloroform was added as an extraction solvent, and the mixture of the aqueous solution and chloroform was heated to allow the conversion to GBL at a temperature of about 60° C. Table 5 shows extraction ratios, and solvent conversion yields of 4-HB thus calculated.

| | | | A- | | | | Sol- |
|---|---|---|---|---|---|---|---|
| | | | mount | | | | vent |
| | Tem- | | of | Initial | | Extrac- | con- |
| | per- | | solvent | 4-HB | GBL | tion | version |
| | ature | | added | concen- | yield | ratio | yield |
| | (° C.) | pH | (fold) | tration | (%) | (%) | (%) |
| Experimental | 60 | 1 | 1 | 11.76 | 90 | 75 | 68 |
| group 1 | | | | | | | |
| Control | Room | 1 | 1 | 11.23 | 58 | 68 | 40 |
| group 1 | tem- | | | | | | |
| | per- | | | | | | |
| | ature | | | | | | |
| Experimental | 60 | 0.5 | 1 | 8.269 | 87 | 74 | 64 |
| group 2 | | | | | | | |
| Control | Room | 0.5 | 1 | 8.269 | 82 | 73 | 60 |
| group 2 | tem- | | | | | | |
| | per- | | | | | | |
| | ature | | | | | | |

*Solvent conversion yield (%) is GBL yield (%)x Extraction ratio(%).

As shown in Table 5, when the results of step 3 of Example 1 are compared to those of Example 2, the conversion of GBL from 4-HB is shown to be significantly improved by heating, and the actual extraction of the converted GBL also was significantly improved.

As described above, according to the one or more of the above exemplary embodiments, a method of producing lactone may efficiently provide lactone from an aqueous solution including hydroxycarboxylic acid or dicarboxylic acid. Also, the method may be efficient in terms of a production process since the method does not need a separate chemical process, such as a salt-treatment or a water-removing process.

It should be understood that the exemplary embodiments described therein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

What is claimed is:

1. A method of producing a lactone, the method comprising
adding an acid to an aqueous solution comprising a hydroxycarboxylic acid or dicarboxylic acid to adjust a pH of the aqueous solution to 4 or lower, whereby the hydroxycarboxylic acid or dicarboxylic acid is converted to a lactone;
adding a water immiscible solvent to the aqueous solution to distribute the lactone to a solvent layer; and
collecting the lactone from the solvent layer.

2. The method of claim 1, wherein the aqueous solution comprising a hydroxycarboxylic acid or dicarboxylic acid is a microorganism culture solution, and the method further comprises culturing a microorganism that produces hydroxycarboxylic acid or dicarboxylic acid to produce the microorganism culture solution.

3. The method of claim 2, wherein culturing of the microorganism is performed at a pH about 6 to about 7 before adding an acid to the aqueous solution to adjust the pH to 4 or lower.

4. The method of claim 2 further comprising filtering the microorganism culture solution and removing retentate.

5. The method of claim 1 further comprising, after adding the water immiscible solvent to the aqueous solution, heating the aqueous solution and water immiscible solvent.

6. The method of claim 5, wherein the aqueous solution is heated to a temperature lower than the boiling point of the solvent.

7. The method of claim 5, wherein collecting the lactone from the solvent layer comprises collecting the solvent layer and distilling the solvent from the solvent layer to separate the lactone from the solvent layer.

8. The method of claim 1, wherein the hydroxycarboxylic acid has a structure of Formula 1,
HO—R1-COOH (Formula 1)
wherein, in Formula 1, R1 is a linear or branched substituted or unsubstituted C1-C20 alkyl group, and
the dicarboxylic acid has a structure of Formula 2,
HOOC—R2-COOH (Formula 2)
wherein, in Formula 2, R2 is a bond or a linear or branched C1-C20 alkyl group.

9. The method of claim 8, wherein the hydroxycarboxylic acid comprises —OH group attached to a carbon at a location of 2, 3, 4, 5, or 6 in Formula 1, and R1 is a linear C1-C10 alkyl group, wherein location refers to the numbering of carbon atoms starting with the carbonyl group carbon of the carboxyl group in a shortest carbon chain length; and the dicarboxylic acid comprises 0, 1, 2, 3, or 4 carbons in a shortest carbon chain that links —COOH group and —COOH group in Formula 2, and R2 is a bond or a linear C1-C8 alkyl group.

10. The method of claim 1, wherein the hydroxycarboxylic acid is 3-hydroxypropionic acid, 4-hydroxybutyric acid, 5-hydroxypentanic acid, or 6-hydroxyhexanoic acid; and the dicarboxylic acid is succinic acid, 2-ethylsuccinic acid, glutaric acid, 2-methylglutaric acid, 2-ethylglutaric acid, adipic acid, 2-methyladipic acid, 3-methyladipic acid, 4-methyladipic acid, 5-methyladipic acid, 2,2-dimethyladipic acid, 3,3-dimethyladipic acid, 2,2,5-trimethyladipic acid, 2,5-dimethyladipic acid, pimelic acid, 2-methylpimelic acid, 2,2-dimethylpimelic acid, 3,3-dimethylpimelic acid, 2,2,5-trimethylpimelic acid, azelic acid, or sebacic acid.

11. The method of claim 1, wherein the pH is in a range of about 0.50 to about 2.00.

12. The method of claim 1, wherein the water immiscible solvent has a water solubility in a range of about 0.05 g/dL to about 1.50 g/dL measured at 20° C. to 25° C. and 1 atm.

13. The method of claim 1, wherein the solvent is dichloromethane or chloroform.

14. The method of claim 1, wherein the lactone is γ-butyrolactone, β-propiolactone, δ-valerolactone, ε-caprolactone, α-angelica lactone, β-angelica lactone, or γ-valerolactone (GVL).

15. The method of claim 1, wherein the aqueous solution comprising a hydroxycarboxylic acid or dicarboxylic acid is a microorganism culture solution comprising 4-hydrobutyric acid, and the method comprises adding an acid to the microorganism culture solution to adjust the pH to 4 or lower.

16. The method of claim 15, wherein the water immiscible solvent is dichloromethane or chloroform.
